(19) United States
(12) Patent Application Publication
(10) Pub. No.: US 2025/0302685 A1
(43) Pub. Date: Oct. 2, 2025

(54) HUMAN WASTE CAPTURING DEVICE

(71) Applicant: Sebastian J. Heredia, Raleigh, NC (US)

(72) Inventor: Sebastian J. Heredia, Raleigh, NC (US)

(21) Appl. No.: 18/619,386

(22) Filed: Mar. 28, 2024

Related U.S. Application Data
(60) Provisional application No. 63/528,901, filed on Jul. 25, 2023.

(51) Int. Cl. A61G 9/00; A61G 9/02

(52) U.S. Cl. CPC A61G 9/003; A61G 9/02; A61G 2200/327; A61G 2200/34

(57) ABSTRACT

A human waste capturing device for hygienic management of bodily waste of a bedbound user includes a bedpan, which is operationally and fluidically couplable to cleaning and waste management modules. The bedpan is partially positionable under buttocks of a user who is bedbound so that a receptacle of the bedpan can receive bodily waste from the user. The cleaning module then selectively directs water and air onto genitals, a perineum, and an anus of the user for rinsing and drying, respectively. The waste management module connects to an air mover so that the bodily waste is urged by passage of air from the bedpan to the waste management module. The waste management module separates the bodily waste from the air so that the bodily waste is segregated for disposal as the air passes through the waste management module to the air mover.

### CROSS-REFERENCE TO RELATED APPLICATIONS

I hereby claim the benefit under 35 U.S.C. Section 119(e) of U.S. Provisional application 63/528,901 filed Jul. 25, 2023.

### STATEMENT REGARDING FEDERALLY SPONSORED RESEARCH OR DEVELOPMENT

### Not Applicable

### THE NAMES OF THE PARTIES TO A JOINT RESEARCH AGREEMENT

### Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

### Not Applicable

### STATEMENT REGARDING PRIOR DISCLOSURES BY THE INVENTOR OR JOINT INVENTOR

### Not Applicable

### BACKGROUND OF THE INVENTION

### (1) Field of the Invention

The disclosure relates to bedpans and bedpan systems and more particularly pertains to a new bedpan and bedpan system for hygienic management of bodily waste of a bedbound user. Hygienic management of such bodily waste has not yet been achieved in an economical and reproducible manner.

### (2) Description of Related Art Including Information Disclosed Under 37 CFR 1.97 and 1.98

The prior art relates to bedpan systems but which do not permit use of a vacuum cleaner to motivate bodily waste from a bedpan to a waste container.

### BRIEF SUMMARY OF THE INVENTION

An embodiment of the disclosure meets the needs presented above by generally comprising a bedpan, which is configured to be operationally and fluidically coupled to both a cleaning module and a waste management module. The bedpan is configured to be partially positioned under buttocks of a user who is bedbound so that a receptacle of the bedpan is configured to receive bodily waste from the user. The cleaning module then selectively directs water and air onto genitals, a perineum, and an anus of the user to rinse and to dry, respectively, the genitals, the perineum, and the anus. The waste management module connects to an air mover so that the bodily waste is urged by passage of air from the bedpan to the waste management module. The waste management module separates the bodily waste from the air so that the bodily waste is segregated for disposal as the air passes through the waste management module to the air mover.

Yet another embodiment of the disclosure includes a waste management system, which comprises a bedpan, as described in the disclosure above, a cleaning module, and a waste management module. A connection assembly attaches the cleaning module to the bedpan allowing the cleaning module to selectively direct water and air onto genitals, a perineum, and an anus of the user to rinse and to dry the genitals, the perineum, and the anus. A tube connector fluidically engaged to the receptacle attaches the bedpan to the waste management module. The waste management module is configured to connect to an air mover to urge the bodily waste and rinse water from the receptacle to the waste management module, wherein the bodily waste is separated from the air.

Yet another embodiment of the disclosure includes a method of managing bodily waste excreted by a bedbound user, which entails provision of a waste management system, according to the disclosure above. Steps of the method include positioning the bedpan partially under buttocks of a user, connecting the waste management module to an air mover, defecating and/or urinating into the receptacle, actuating the cleaning module, and urging of the bodily waste and rinse water from the receptacle to the waste management module by passage of air.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a human waste capturing device according to an embodiment of the disclosure.

FIG. 2 is an isometric perspective view of an embodiment of the disclosure.

FIG. 3 is a side view of an embodiment of the disclosure.

FIG. 4 is a detail isometric perspective view of an embodiment of the disclosure.

FIG. 5 is a rear detail view of an embodiment of the disclosure.

FIG. 6 is a cross-sectional view of an embodiment of the disclosure.

FIG. 7 is a detail isometric perspective view of an embodiment of the disclosure.

FIG. 8 is a cross-sectional view of an embodiment of the disclosure.

FIG. 9 is a cross-sectional view of an embodiment of the disclosure.

FIG. 10 is a detail view of an embodiment of the disclosure.

FIG. 11 is an isometric perspective view of an embodiment of the disclosure.

FIG. 12 is an isometric perspective view of an embodiment of the disclosure.

FIG. 13 is an isometric perspective view of an embodiment of the disclosure.

FIG. 14 is an isometric perspective view of an embodiment of the disclosure.

FIG. 15 is a rear view of an embodiment of the disclosure.

FIG. 16 is a cross-sectional view of an embodiment of the disclosure.

FIG. 17 is a cross-sectional view of an embodiment of the disclosure.

FIG. 18 is a cross-sectional view of an embodiment of the disclosure.

FIG. 19 is a flow diagram for a method utilizing an embodiment of the disclosure.

### DETAILED DESCRIPTION OF THE INVENTION

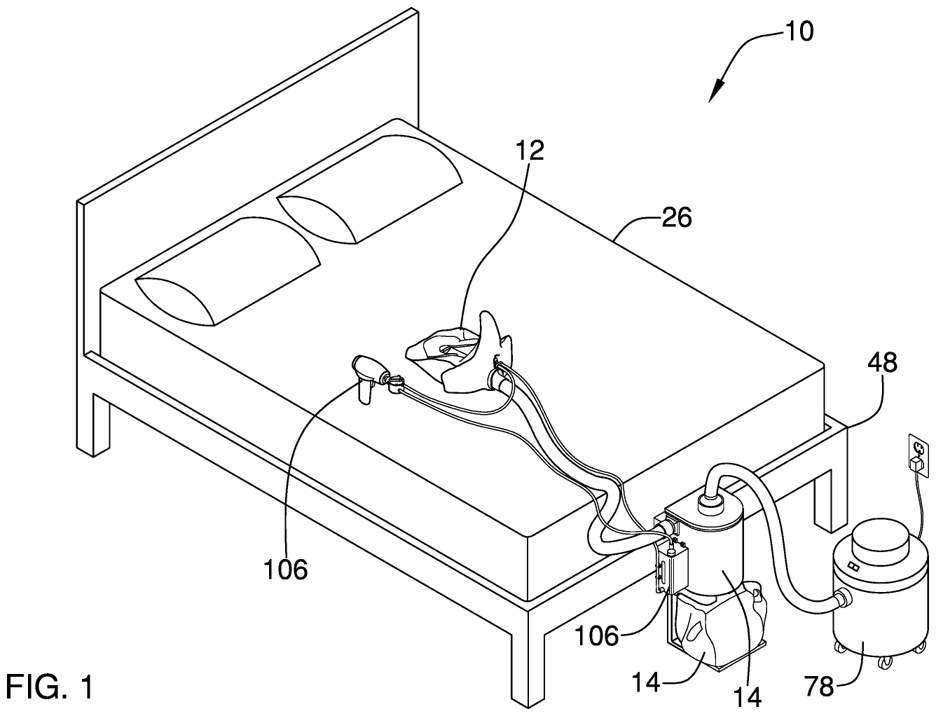

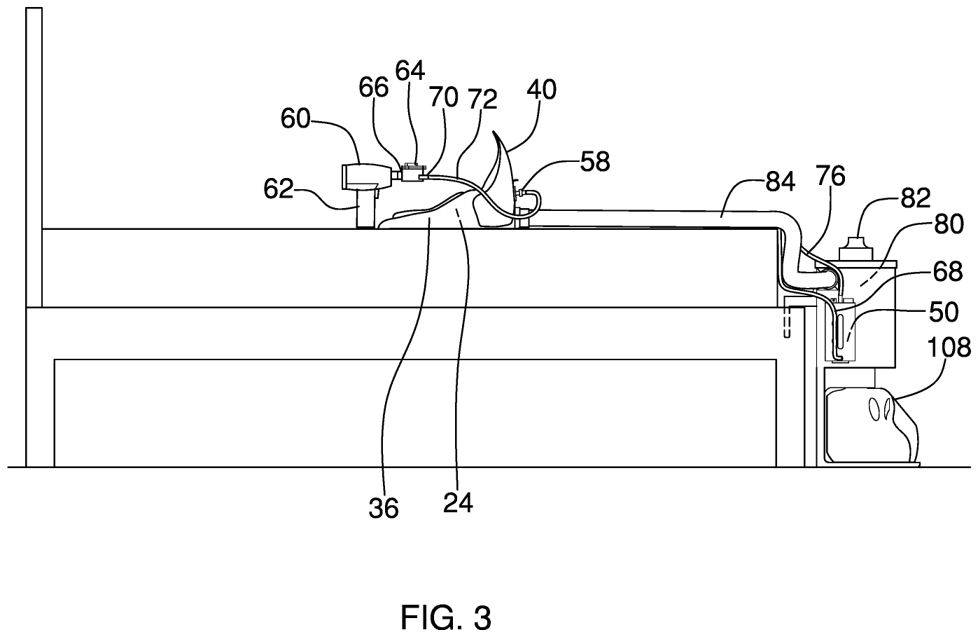

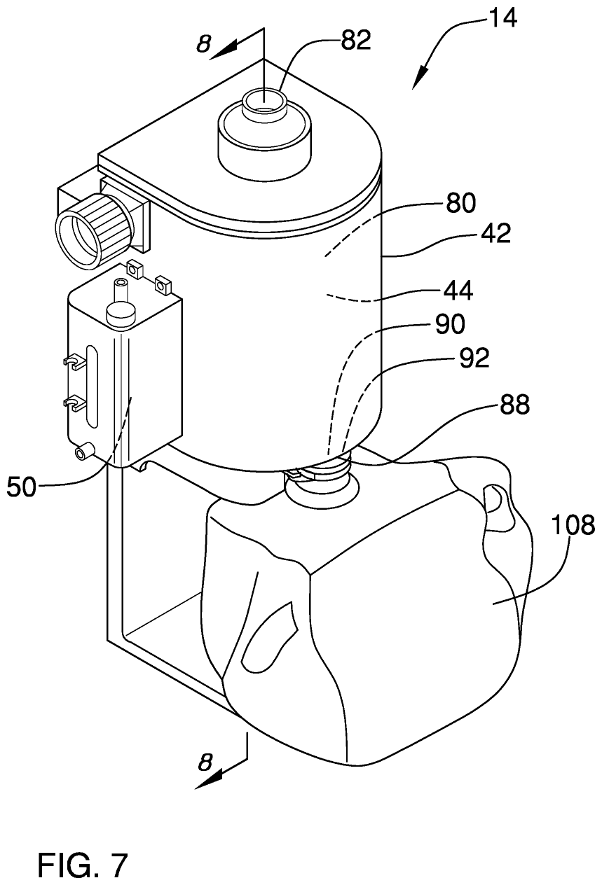

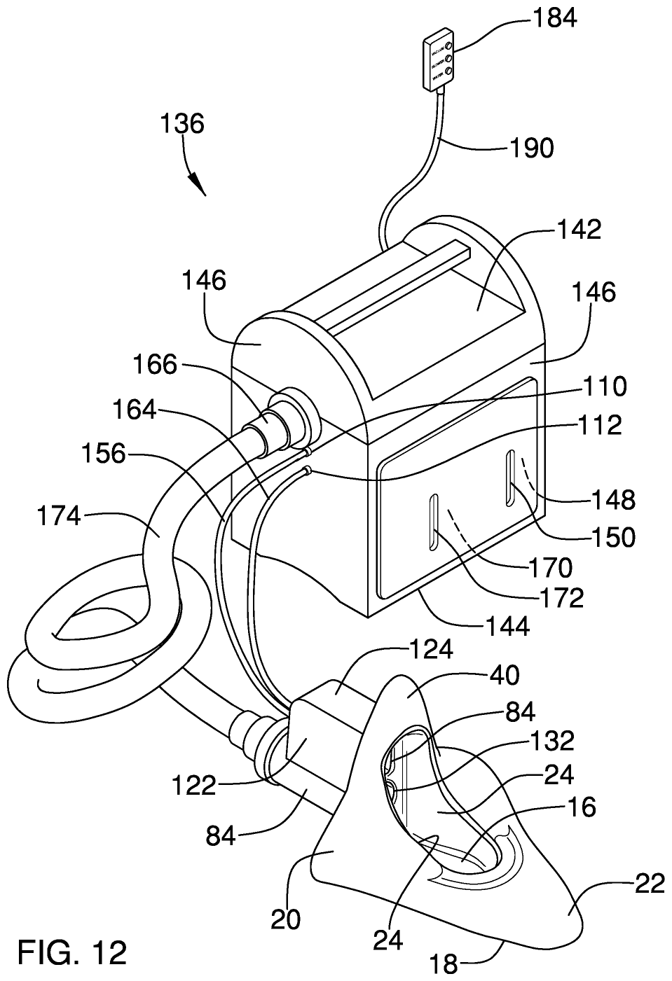

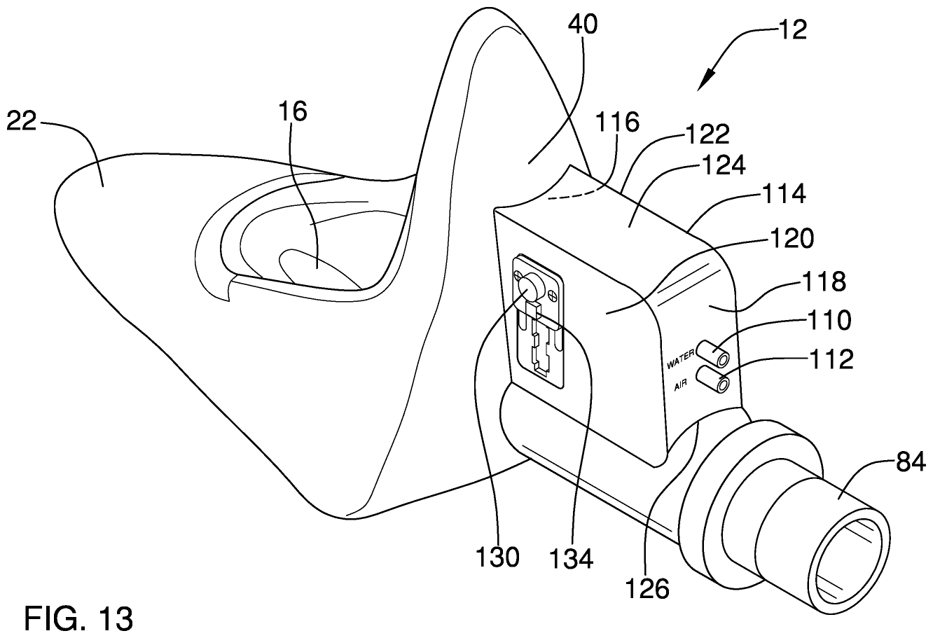

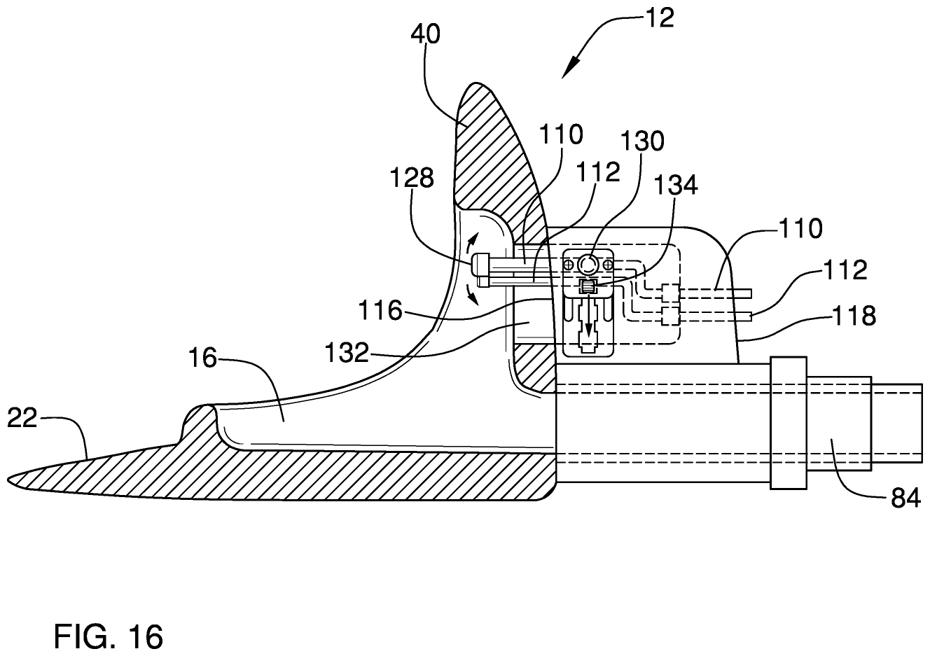

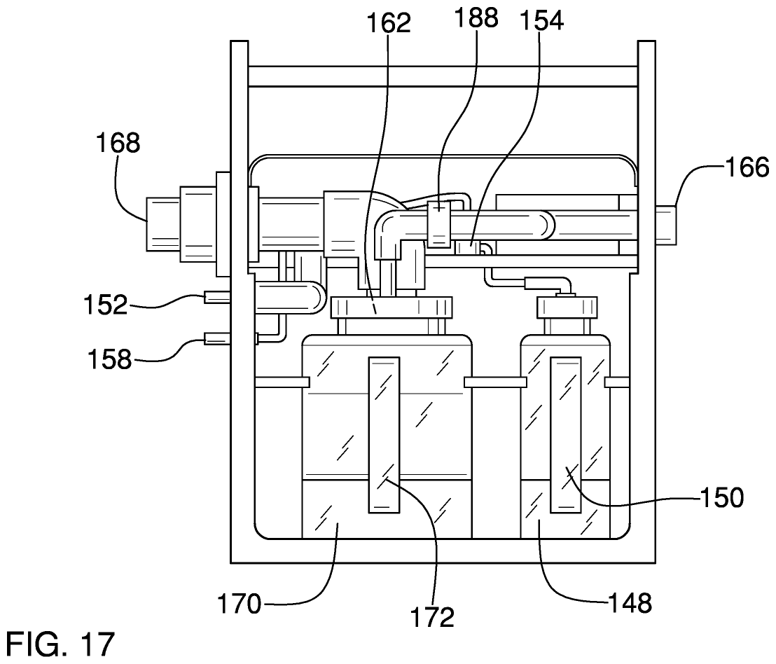

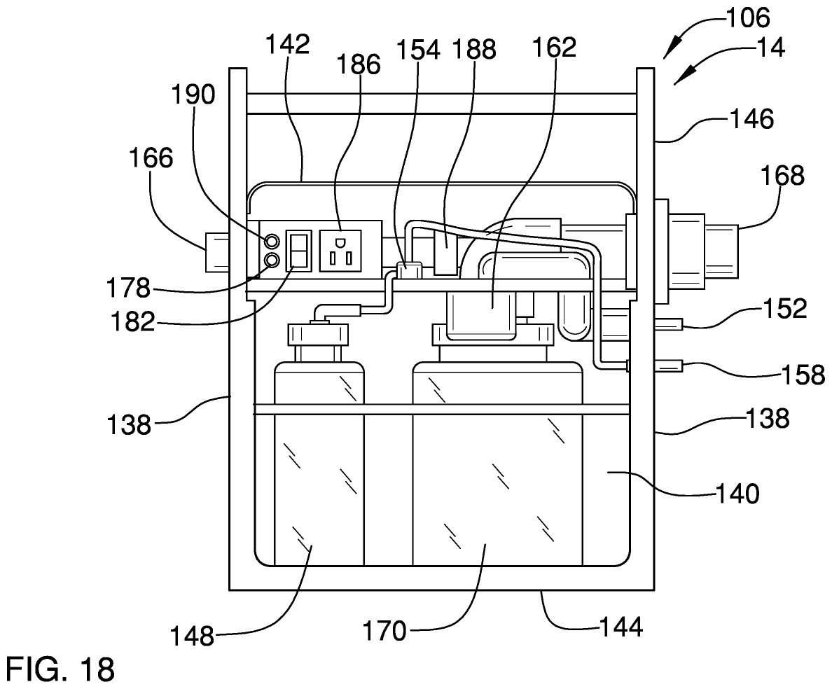

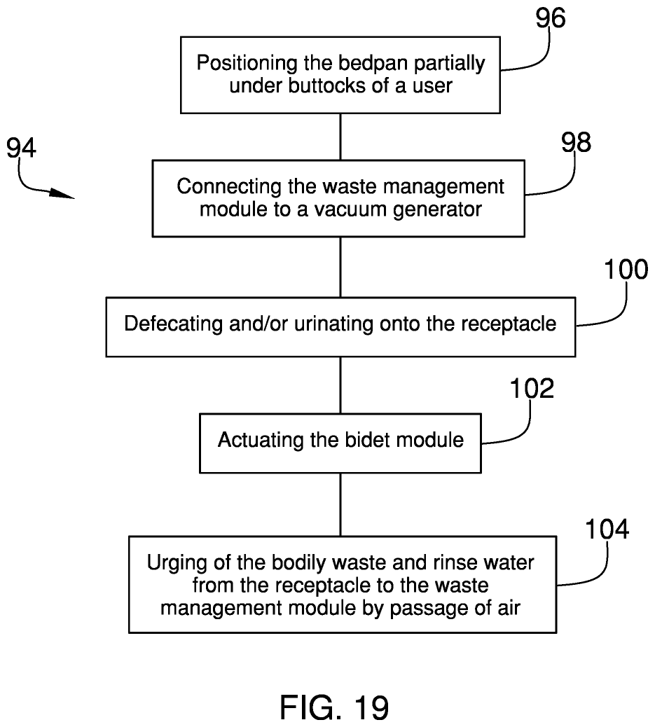

With reference now to the drawings, and in particular to FIGS. 1 through 19 thereof, a new bedpan system embodying the principles and concepts of an embodiment of the disclosure and generally designated by the reference numeral 10 will be described.

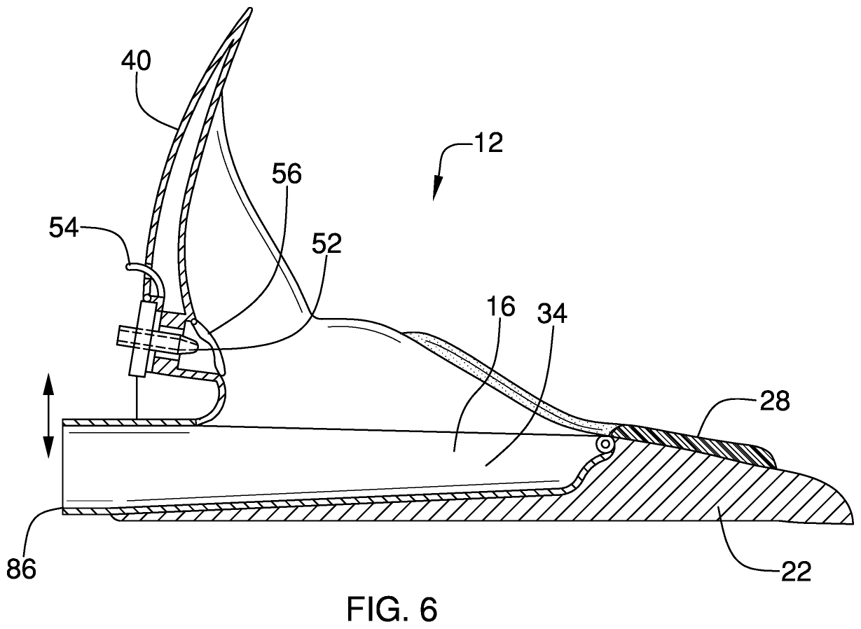

As best illustrated in FIGS. 1 through 19, the human waste capturing device 10 generally comprises a bedpan 12, which is configured to be operationally engaged to a cleaning module 106 and to a waste management module 14 by means of a connection assembly 58, which is attached to the bedpan 12. The bedpan 12 is configured to be partially positioned under buttocks of a user who is bedbound so that a receptacle 16 of the bedpan 12 is configured to receive bodily waste from the user. The bedpan 12 comprises a first piece 18 and a second piece 20. The first piece 18 comprises a rearward section 22 and a pair of inner panels 24. The rearward section 22 is wedge shaped, as is shown in FIG. 6, so that the rearward section 22 is configured for insertion between a mattress 26 and the buttocks of the user.

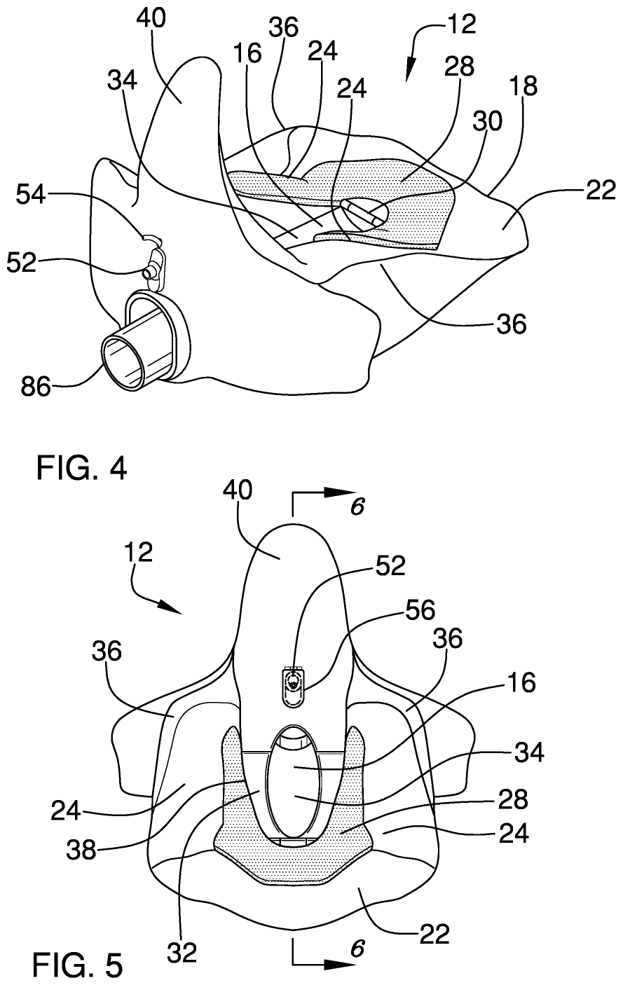

The pair of inner panels 24 is attached to and extends forwardly from the rearward section 22 so that the inner panels 24 bracket posterior thighs of the user. Padding 28 may be attached to the first piece 18 to cushion a weight bearing area of the first piece 18 to increase comfort of the user. The padding 28 typically would comprise silicone, rubber, or the like. The second piece 20, which comprises the receptacle 16, may be hingedly attached to a front edge 30 of the rearward section 22 of the first piece 18, as is shown in FIG. 4. The first piece 18 thus is configured to hinge relative the second piece 20 upon positioning of the buttocks of the user on the first piece 18. The receptacle 16 thereby remains substantially level or tilts downwardly away from the user, thus limiting a risk of spillage of bodily waste.

The second piece 20 comprises a bottom panel 32, which has an ovoid recess 34 positioned therein to define the receptacle 16. Each outer panel 36 of a pair of outer panels 36 is attached to and extends angularly and outwardly from a respective opposed edge 38 of the bottom panel 32. The outer panels 36 abut and bracket the inner panels 24 and slide over one another as the first piece 18 hinges relative the second piece 20. A front panel 40 is attached to and extends upwardly from the bottom panel 32. The front panel 40 also is attached to and extends between the outer panels 36. As is shown in the figures, the connection assembly 58 is attached to the front panel 40. The front panel 40 is dimensionally taller than the outer panels 36 thus is configured to direct urine to the receptacle 16, essentially serving as a splashguard. The front panel 40 is convexly arcuate distal from the bottom panel 32 to increase user comfort during use of the human waste capturing device 10. While the bottom panel 32, outer panels 36, and front panel 40 are is described separately for descriptive purposes, the present invention anticipates them comprising a unitary structure. Typically, the first piece 18 and the second piece 20 of the bedpan 12 would be molded to conform to and to support the user during use of the bedpan 12.

In another embodiment, as is shown in FIG. 13, the first piece 18 and the second piece 20 comprising a unitary structure so that the receptacle 16 is not hingable relative to the rearward section 22 of the first piece 18. Rather, the first piece 18 comprises resiliently compressible material, such as rubber, elastomer, or the like. Upon positioning of the buttocks of the user on the first piece 18, the receptacle 16 remains substantially level or tilts downwardly away from the user, thereby limiting a risk of spillage of bodily waste. The present invention also anticipates a liner that can be removably positioned within the bedpan 12. The liner being removeable and washable facilitates good hygiene.

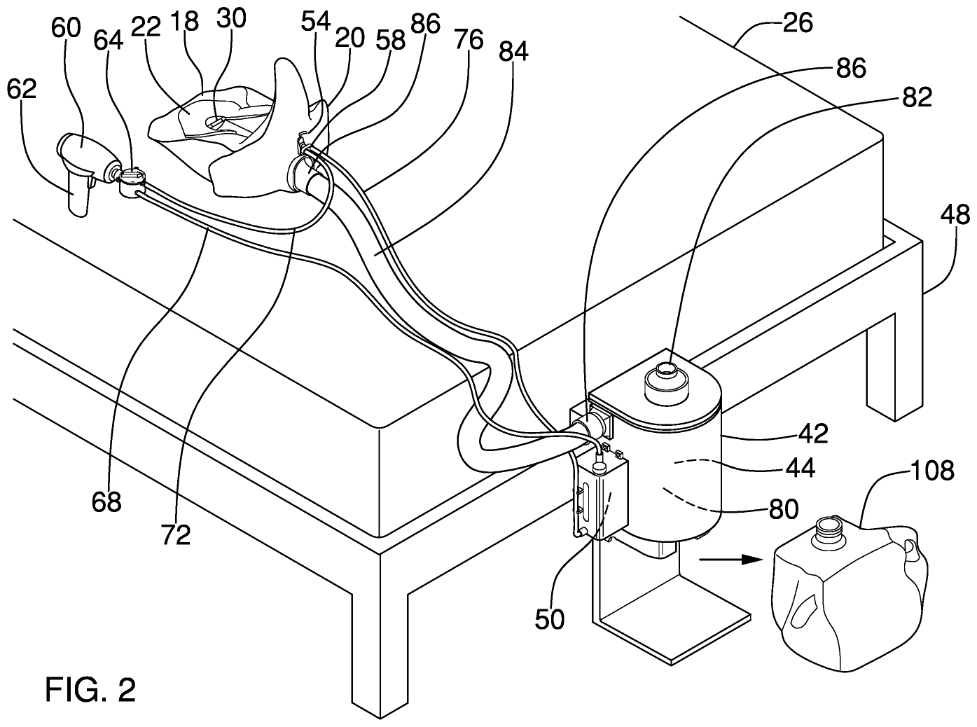

The present invention anticipates a waste capturing system 136 comprising the human waste capturing device 10. The waste capturing system 136 may comprise a utility housing 42, which defines an interior space 44 and which is depicted in FIGS. 1-3 and 7-9. A bracket 46 may be attached to the utility housing 42 and be configured to mount the utility housing 42 to a bed 48, as is shown in FIG. 2. The present invention also anticipates the utility housing 42 being configured to simply rest on a floor proximate to the bed 48.

The cleaning module 106 is attached to the connection assembly 58 and is configured to selectively direct water and air onto genitals, a perineum, and an anus of the user. The cleaning module 106 thus is configured to rinse and to dry the genitals, the perineum, and the anus, with water and air, respectively, thereby enhancing hygiene for the user.

The cleaning module 106 comprises a reservoir 50, which is fillable with water and which is attached to the utility housing 42. The present invention also anticipates the reservoir 50 being positioned in the interior space 44. A spray nozzle 52 is attached to the bedpan 12 and is configured to direct the water and the air onto the genitals, the perineum, and the anus. As is shown in FIG. 5, the spray nozzle 52 is attached to the front panel 40. The spray nozzle 52 may be hingedly attached to the bedpan 12, as is shown in FIG. 6, so that the directing of the water and the air is adjustable. A lever 54 is attached to the bedpan 12, is operationally engaged to the spray nozzle 52, and is configured to be levered to selectively direct the water and the air that are emitted by the spray nozzle 52. Other adjustment means for hinging the spray nozzle 52 also are anticipated, such as, but not limited to, grooved wheels, slide tabs, or the like.

A flap 56 is hingedly attached to the bedpan 12 and is positioned over the spray nozzle 52. The flap 56 is configured to shield the spray nozzle 52 from the bodily waste. The flap 56 is deflectable by the water and the air so that the water and the air are directed onto the genitals, the perineum, and the anus of the user. The flap 56 returns to a covering position over the spray nozzle 52 upon termination of the flow of water or the flow of air.

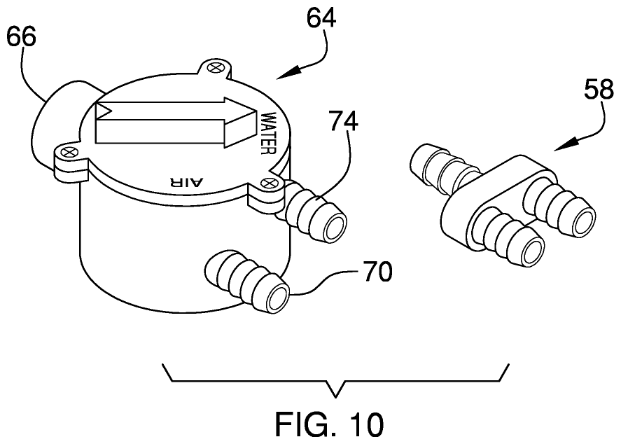

The connection assembly 58, one embodiment of which is shown in detail to the right in FIG. 10, man comprise a three-way connector 192 that is attached to the spray nozzle 52. The cleaning module 106 comprises an air blower 60. A grip 62 is attached to the air blower 60 and is configured to be grasped in a hand of the user to hold the air blower 60. The air blower 60 also is anticipated to be attached to the utility housing 42 where it would be operated by a caregiver. Air blower in the context of this disclosure should be interpreted to mean any air moving device that utilizes a pump, a fan, or the like to move air. Examples include hair driers, which may supply heated air to enhance drying, air pumps, or the like. The present invention anticipates the air blower 60 being plug-in type, as is shown in the figures, or battery powered so that the human waste capturing device 10 does not require access to an electrical circuit.

A switching valve 64, which is shown in detail to the left in FIG. 10, has an inlet port 66 that is attached to the air blower 60. A first air hose 68 is attached to and extends between a first outlet port 70 of the switching valve 64 and the reservoir 50. A second air hose 72 is attached to and extends between a second outlet port 74 of the switching valve 64 and the three-way connector 192. A water hose 76 is attached to and extends between the reservoir 50 and the three-way connector 192. The air blower 60 is configured to be actuated to blow air and the switching valve 64 is configured to be selectively switched so that the air is directed through the first air hose 68 to the reservoir 50, thereby forcing water through the water hose 76 to the spray nozzle 52, or through the second air hose 72 to the spray nozzle 52, to rinse or to dry the user, respectively.

The waste management module 14 is in fluidic communication with the bedpan 12 and is configured to connect to an air mover 78 so that the bodily waste is urged by passage of air from the bedpan 12 to the waste management module 14. The waste management module 14 is configured to separate the bodily waste from the air so that the bodily waste is segregated for disposal as the air passes through the waste management module 14 to the air mover 78. The waste management module 14 comprises a separator 80, which is attached to the utility housing 42 and which is positioned in the interior space 44.

A vacuum connector 82 is attached to the utility housing 42 so that the vacuum connector 82 is in fluidic communication with the separator 80. The vacuum connector 82 is configured to connect to the air mover 78 and enables any standard vacuum cleaner having a hose to function as the air mover 78. The present invention also anticipates adapters, which would be selectively attachable to the vacuum connector 82 and which would enable connection to air movers 78 installed in hospitals, nursing homes, long term care facilities, or the like.

A waste tube 84 is attached to and extends between the bedpan 12 and the utility housing 42 so that the waste tube 84 is in fluidic communication with both the separator 80 and the receptacle 16. As is shown in FIG. 2, the waste tube 84 is selectively connectable to the utility housing 42 and to the bedpan 12 using tube connectors 86 that are attached singly to the utility housing 42 and to the bedpan 12. A waste container 108 is positioned below the separator 80 and is configured to receive the bodily waste that is separated from the air. The bodily waste and rinse water then can be dumped from the waste container 108 into a toilet.

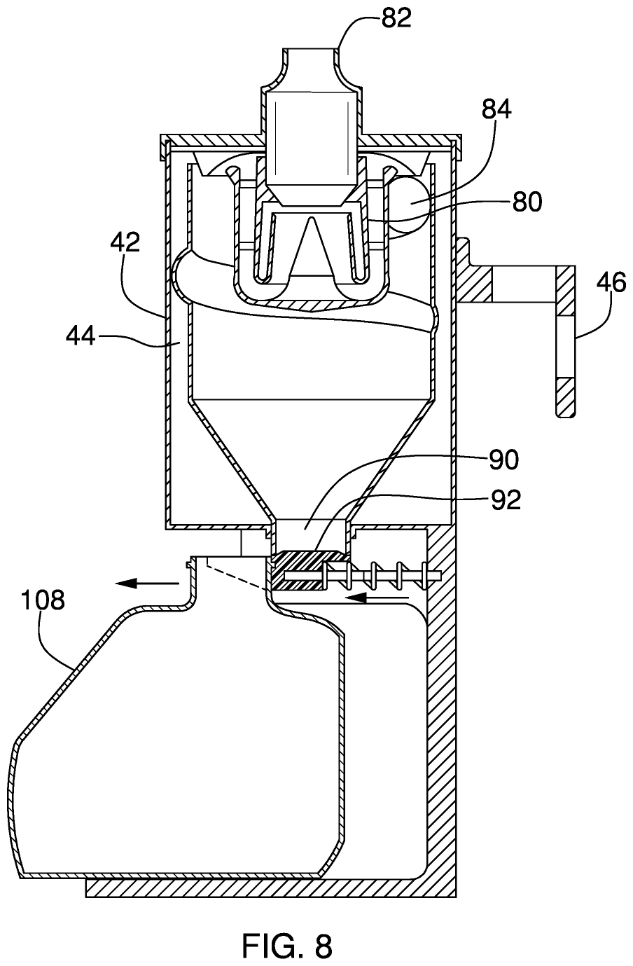

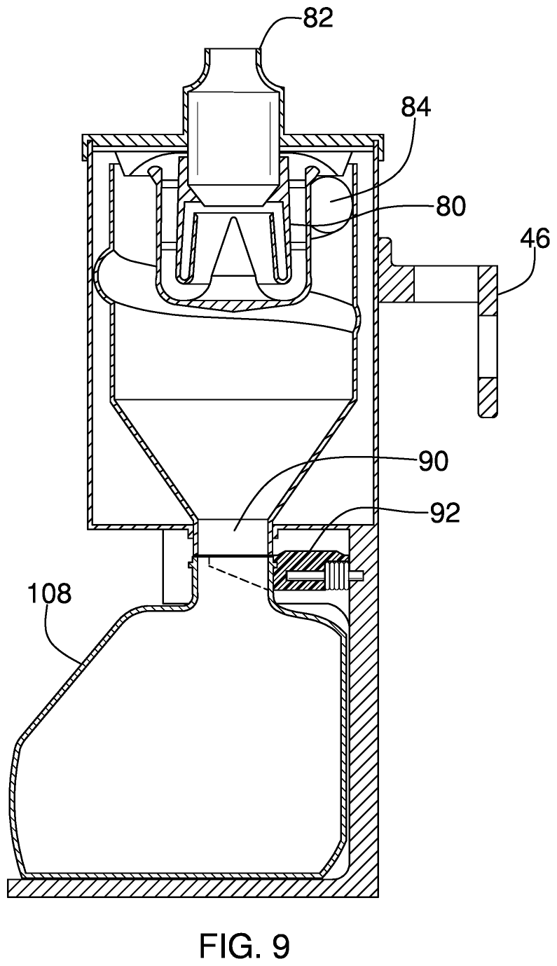

A bottom 88 of the utility housing 42 has a discharge port 90 positioned therein. The waste container 108 is selectively attachable to the utility housing 42 so that the waste container 108 is in fluidic communication with the interior space 44 via the discharge port 90. A trapdoor 92 is attached to the utility housing 42 and is positioned to selectively cover the discharge port 90. The trapdoor 92 is urged to an open configuration concurrently with attachment of the waste container 108, as is shown in FIG. 9. The trapdoor 92 closes the discharge port 90 upon detachment of the waste container 108, as is shown in FIG. 8. Other means of selectively opening and closing the discharge port 90 are anticipated, such as, but not limited to, sanitary valves, diaphragm valves, or the like.

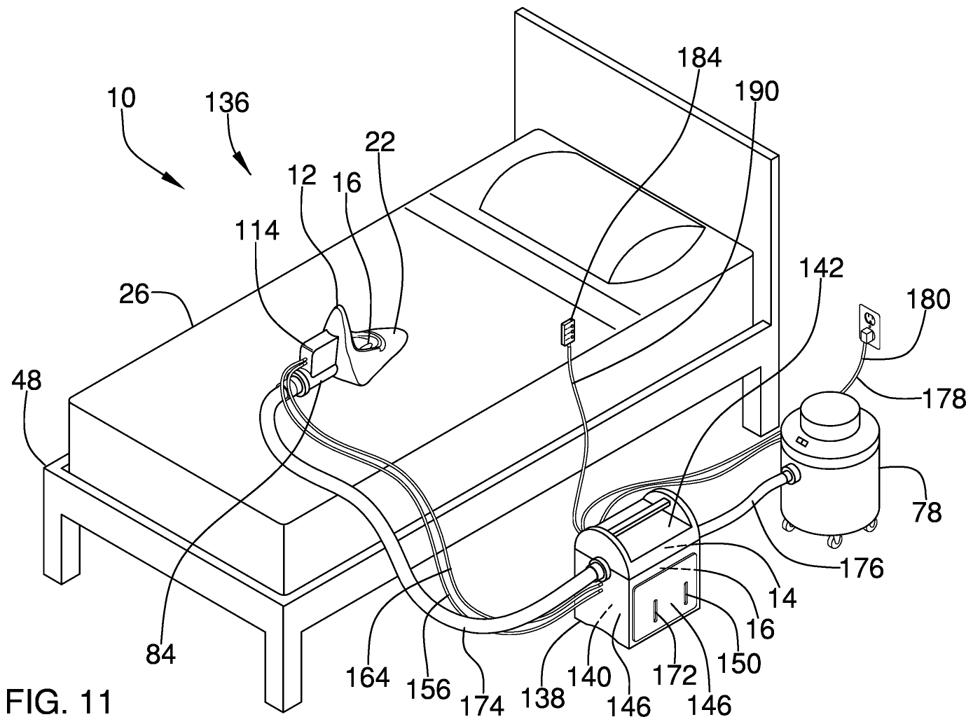

Another embodiment of the waste capturing system 136 is shown in FIGS. 11, 12, 14, 17, and 18, wherein the cleaning module 106 and the waste management module 14 are housed within a module housing 138, which defines an internal space 140. The bedpan 12 used in this embodiment is shown in FIG. 13 and include a connection housing 114 having a first end 116, a second end 118, a first side 120, a second side 122, a top 124, and a bottom 126. The first end 116 is attached to the front panel 40. The connection assembly 58 of this embodiment comprises a liquid connector 110 and an air connector 112, which are attached to the second end 118 of the connection housing 114. The liquid connector 110 is configured to connect the bedpan 12 to the source of pressurized water for rinsing the genitals, the perineum, and the anus of the user. The air connector 112 is configured to connect the bedpan 12 to the air blower 60 to dry the genitals, the perineum, and the anus of the user. The tube connector 86 is attached to the bottom 126 of the connection housing 114 and is configured to fluidically connect the receptacle 16 to the air mover 78 to urge the bodily waste and the rinse water from the receptacle 16 and away from the user.

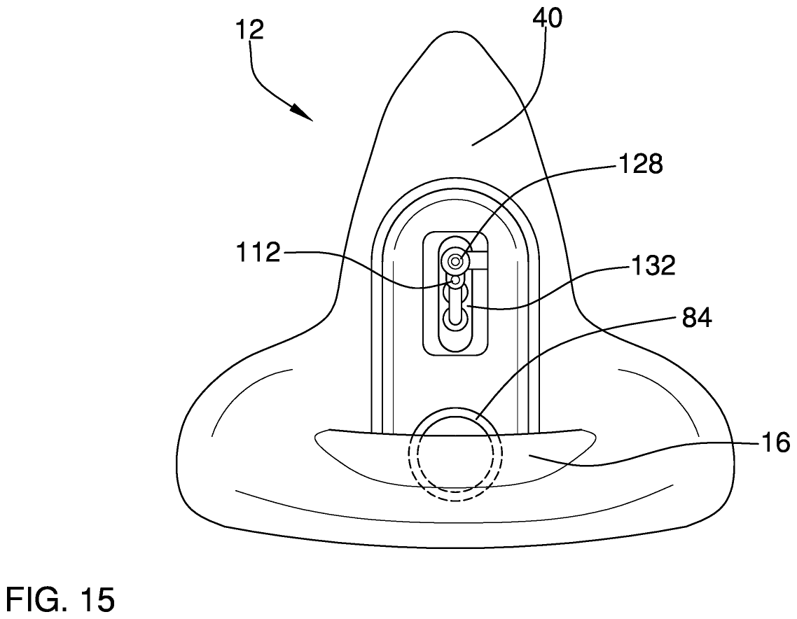

As is shown in FIG. 15, the first end 116 of the connection housing 114 has a vertical slot 132 positioned therein. The vertical slot 132 extends through the front panel 40. The liquid connector 110 and the air connector 112 extend through the connection housing 114 and protrude through the vertical slot 132. A sprayer nozzle 128 is attached to the liquid connector 110 and positioned in the bedpan 12. A knob 130 is rotationally attached to the first side 120 of the connection housing 114 and is operationally engaged to the sprayer nozzle 128. The knob 130 thus is configured to be turned to selectively direct the water emitted by the sprayer nozzle 128. The liquid connector 110 and the air connector 112 are selectively vertically positionable within the vertical slot 132. A slider 134 attached to the first side 120 of the connection housing 114 and is operationally engaged to the liquid connector 110 and the air connector 112 to adjust a position of the liquid connector 110 and the air connector 112 within the vertical slot 132, thereby facilitating their optimal positioning for cleaning and drying the user.

The module housing 138 has a top 142, a bottom 144, and a plurality of sidewalls 146, the sidewalls 146 of which extend between the top 142 and the bottom 144. The cleaning module 106 of this embodiment comprises a water tank 148, which is fillable with water and selectively positionable in the module housing 138. A first window 150 may be positioned is a respective sidewall 146 adjacent to the water tank 148 to monitor a level of water in the water tank 148. A water outlet 152 extends through a respective sidewall 146 of the module housing 138. A water pump 154 is attached to and is positioned in the module housing 138. The water pump 154 being in fluidic communication with the water tank 148 and the water outlet 152. A water tube 156 is used to connect the water outlet 152 to the liquid connector 110 of the bedpan 12, thereby enabling rinsing of the user.

An air outlet 158 and an air inlet 160 extend through a respective sidewalls 146 of the module housing 138. An air pump 162, which is attached to and which is positioned in the module housing 138, is in fluidic communication with the air outlet 158 and the air inlet 160. An air tube 164 is to connect the air outlet 158 to the air connector 112 of the bedpan 12, thereby enabling air-drying of the user.

The waste management module 14 of this embodiment comprises a vacuum inlet pipe 166 and a waste inlet pipe 168, which are positioned in respective sidewalls 146 of the module housing 138 and which curve toward the bottom 144 of the module housing 138. A waste tank 170 is selectively positionable in the module housing 138 and is attachable to the waste inlet pipe 168 and to the vacuum inlet pipe 166. A second window 172 may be positioned is a respective sidewall 146 adjacent to the waste tank 170 to monitor a level of waste in the waste tank 170. A waste hose 174 is selectively attachable the waste inlet pipe 168 and the waste tube 84 of the bedpan 12, thereby allowing the waste and the rinse water to flow from the reservoir 16 to the waste tank 170. A vacuum hose 176 is selectively attachable the vacuum inlet pipe 166 and is configured to attach to the air mover 78, thereby allowing air flowing through the reservoir to urge the waste and rinse water to the waste tank 170. Typically, a filter 188 will be positioned in the vacuum inlet pipe 166 so that particulate matter in the air passing to the air mover 78 is removed.

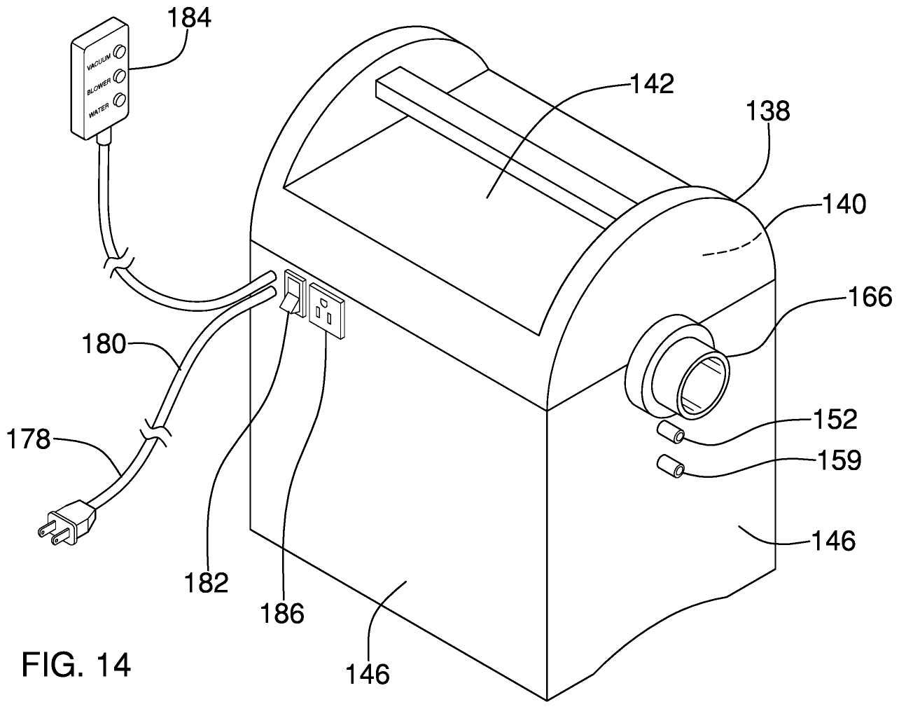

A power supply 178 is attached to the module housing 138 and is operationally engaged to the air pump 162 and the water pump 154. As is shown in FIGS. 11 and 14, the power supply 178 comprises a power cord 180, although the present invention also anticipates the waste capturing system 136 being battery powered. A switch 182 is attached to the module housing 138 and is operationally engaged to the power supply 178, the air pump 162, and the water pump 154. The switch 182 thus can selectively power on the air pump 162 and the water pump 154.

The waste capturing system 136 may comprise a controller 184, which is operationally engaged to the water pump 154 and the air pump 162 to selectively actuate the cleaning module 106. As is shown in FIG. 11, the controller 184 is accessible to the user positioned on the mattress 26, thereby allowing the user to control the cleaning module 106. A power outlet 186 may be attached to the module housing 138 and operationally engaged to the power supply 178 and the controller 184, as is shown in FIG. 14. The air mover 78 can be powered via the power supply 178 and selectively actuated by the controller 184. The controller 184 may be wiredly engaged to the power outlet 186, the water pump 154, and the air pump 162, as is shown in FIG. 11 wherein a remote cord 190 is used, although the present invention also anticipates the controller 184 being wireless.

In use, the human waste capturing device 12 enables a method of managing bodily waste excreted by a bedbound user 94, which entails provision of a human waste capturing device 12, according to the specification above. A first step 96 of the method 94 is positioning the bedpan 12 partially under buttocks of a user who is bedbound so that the receptacle 16 is positioned to receive bodily waste from the user. A second step 98 of the method 94 is connecting the waste management module 14 to an air mover 78. A third step 100 of the method 94 is defecating and/or urinating into the receptacle 16. A fourth step 102 of the method 94 is actuating the cleaning module 106 to selectively direct water and air onto the genitals, the perineum, and the anus of the user to rinse and to dry the genitals, the perineum, and the anus. A fifth step 104 of the method 94 is urging of the bodily waste and rinse water from the receptacle 16 to the waste management module 14 by passage of air.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A human waste capturing device comprising:
a bedpan having a receptacle and being configured to be partially positioned under buttocks of a user who is bedbound, wherein the receptacle is configured to receive bodily waste from the user;
a connection assembly attached to the bedpan and being configured to connect the bedpan to a source of pressurized water and an air blower for rinsing and drying, respectively, genitals, a perineum, and an anus of the user; and
a tube connector attached to the bedpan and being configured to fluidically connect the receptacle to an air mover for urging the bodily waste and rinse water from the receptacle and away from the user.

2. The human waste capturing device of claim 1, wherein the bedpan comprises:
a first piece comprising:
a rearward section, the rearward section being wedge shaped, such that the rearward section is configured for insertion between a mattress and the buttocks of the user; and
a pair of inner panels attached to and extending forwardly from the rearward section, such that the inner panels of the pair of inner panels bracket posterior thighs of the user; and
a second piece comprising the receptacle being hingedly attached to a front edge of the rearward section of the first piece, wherein the first piece is configured to hinge relative the second piece upon positioning of the buttocks of the user on the first piece, such that the receptacle remains substantially level or tilts downwardly away from the user, thereby limiting a risk of spillage of bodily waste.

3. The human waste capturing device of claim 2, wherein the second piece comprises:
a bottom panel having an ovoid recess positioned therein defining the receptacle;
a pair of outer panels, each outer panel of the pair of outer panels being attached to and extending angularly and outwardly from a respective opposed edge of the bottom panel, such that the outer panels of the pair of outer panels abut and bracket the inner panels of the pair of inner panels; and
a front panel attached to and extending upwardly from the bottom panel, the front panel being attached to and extending between the outer panels of the pair of outer panels, the front panel being dimensionally taller than the pair of outer panels, the front panel being convexly arcuate distal from the bottom panel, wherein the front panel is configured to direct urine to the receptacle.

4. The human waste capturing device of claim 1, further including:
the connection assembly comprising a three-way connector;
a spray nozzle being hingedly attached to the bedpan and fluidically engaged to the three-way connector; and
a lever attached to the bedpan and operationally engaged to the spray nozzle, wherein the lever is configured to be levered to selectively direct the water and the air emitted by the spray nozzle.

5. The human waste capturing device of claim 4, further including a flap attached to the bedpan and positionable over the spray nozzle, wherein the flap is configured to shield the spray nozzle from the bodily waste, the flap being deflectable by the water and the air, such that the water and the air are directed onto the genitals, the perineum, and the anus of the user.

6. The human waste capturing device of claim 4, wherein:
the three-way connector is configured to connect the bedpan to a source of pressurized water and an air blower that are available in a care facility; and
the tube connector is configured to fluidically connect the receptacle to a waste management module that is available in the care facility.

7. The human waste capturing device of claim 2, further including:
the second piece comprising:
a bottom panel having an ovoid recess positioned therein defining the receptacle; and
a front panel being attached to and extending upwardly from the bottom panel, the front panel being attached to and extending between the outer panels of the pair of outer panels, the front panel being dimensionally taller than the pair of outer panels, the front panel being convexly arcuate distal from the bottom panel, wherein the front panel is configured to direct urine to the receptacle;
the first piece and the second piece comprising a unitary structure such that the receptacle is not hingable relative to the rearward section of the first piece, the first piece comprising resiliently compressible material such that, upon positioning of the buttocks of the user on the first piece, the receptacle remains substantially level or tilts downwardly away from the user, thereby limiting a risk of spillage of bodily waste.

8. The human waste capturing device of claim 7, further including:
a connection housing having a first end, a second end, a first side, a second side, a top, and a bottom, the first end being attached to the front panel;
the connection assembly comprising:
a liquid connector being attached to the second end of the connection housing and being configured to connect the bedpan to the source of pressurized water for rinsing the genitals, the perineum, and the anus of the user; and
an air connector being attached to the second end of the connection housing and being configured to connect the bedpan to the air blower for drying the genitals, the perineum, and the anus of the user; and
the tube connector being attached to the bottom of the connection housing and being configured to fluidically connect the receptacle to the air mover for urging the bodily waste and the rinse water from the receptacle and away from the user.

9. The human waste capturing device of claim 8, wherein:
the first end of the connection housing has a vertical slot positioned therein, the vertical slot extending through the front panel; and
the liquid connector and the air connector extend through the connection housing and protrude through the vertical slot.

10. The human waste capturing device of claim 9, further including:
a sprayer nozzle being attached to the liquid connector and positioned in the bedpan; and
a knob being rotationally attached to the first side of the connection housing and being operationally engaged to the sprayer nozzle, wherein the knob is configured to be turned to selectively direct the water emitted by the sprayer nozzle.

11. The human waste capturing device of claim 9, further including:
the liquid connector and the air connector being selectively vertically positionable within the vertical slot; and
a slider being attached to the first side of the connection housing and being operationally engaged to the liquid connector and the air connector for adjusting a position of the liquid connector and the air connector within the vertical slot.

12. The human waste capturing device of claim 8, wherein:
the liquid connector is configured to connect the bedpan to a source of pressurized water that is available in a care facility;
the air connector is configured to connect the bedpan to an air blower that is available in a care facility; and
the tube connector is configured to fluidically connect the receptacle to a waste management module that is available in the care facility.

13. A waste capturing system comprising:
a bedpan having a receptacle and being configured to be partially positioned under buttocks of a user who is bedbound, wherein the receptacle is configured to receive bodily waste from the user;
a connection assembly being attached to the bedpan; and
a tube connector being attached to the bedpan and being fluidically engaged to the receptacle;
a cleaning module being attached to the connection assembly and being configured to selectively direct water and air onto genitals, a perineum, and an anus of the user, wherein the cleaning module is configured to rinse and to dry the genitals, the perineum, and the anus, with water and air, respectively; and
a waste management module being attached to the tube connector, in fluidic communication with the bedpan, and being configured to connect to an air mover for urging the bodily waste and rinse water from the receptacle to the waste management module, the waste management module being configured to separate the bodily waste from the air, such that the bodily waste is segregated for disposal as the air passes through the waste management module to the air mover.

14. The waste capturing system of claim 13, further including:
the connection assembly comprising a three-way connector;
a utility housing defining an interior space;
the cleaning module comprising:
a reservoir fillable with water and attached to the utility housing;
an air blower;
a switching valve having an inlet port attached to the air blower;
a first air hose attached to and extending between a first outlet port of the switching valve and the reservoir;
a second air hose attached to and extending between a second outlet port of the switching valve and the three-way connector; and
a water hose attached to and extending between the reservoir and the three-way connector, wherein the air blower is configured to be actuated to blow air and the switching valve is configured to be selectively switched such that the air is directed through the first air hose to the reservoir so that water is forced through the water hose to the spray nozzle, or through the second air hose to the spray nozzle, to rinse or to dry the user, respectively; and
the waste management module comprising:
a separator attached to the utility housing and positioned in the interior space;
a vacuum connector attached to the utility housing such that the vacuum connector is in fluidic communication with the separator, the vacuum connector being configured to connect to the air mover;
a waste tube attached to and extending between the bedpan and the utility housing, such the waste tube is in fluidic communication with both the separator and the receptacle; and
a waste receptacle positioned below the separator, wherein the waste receptacle is configured to receive the bodily waste that is separated from the air.

15. The waste capturing system of claim 13, further including the bedpan comprising:
a first piece comprising:
a rearward section, the rearward section being wedge shaped, such that the rearward section is configured for insertion between a mattress and the buttocks of the user; and
a pair of inner panels attached to and extending forwardly from the rearward section, such that the inner panels of the pair of inner panels bracket posterior thighs of the user; and
a second piece comprising:
a bottom panel being attached to a front edge of the rearward section of the first piece, the bottom panel having an ovoid recess positioned therein defining the receptacle; and
a front panel being attached to and extending upwardly from the bottom panel, the front panel being attached to and extending between the outer panels of the pair of outer panels, the front panel being dimensionally taller than the pair of outer panels, the front panel being convexly arcuate distal from the bottom panel, wherein the front panel is configured to direct urine to the receptacle; and
the first piece and the second piece comprising a unitary structure, the first piece comprising resiliently compressible material such that, upon positioning of the buttocks of the user on the first piece, the receptacle remains substantially level or tilts downwardly away from the user, thereby limiting a risk of spillage of bodily waste.

16. The waste capturing system of claim 15, further including:
a module housing comprising a top, a bottom, and a plurality of sidewalls extending between the top and the bottom;
the cleaning module comprising:
a water tank fillable with water and selectively positionable in the module housing;
a water outlet extending through a respective sidewall of the module housing;
a water pump attached to and positioned in the module housing, the water pump being in fluidic communication with the water tank and the water outlet;
an air outlet extending through a respective sidewall of the module housing;
an air inlet extending through a respective sidewall of the module housing;
an air pump attached to and positioned in the module housing, the air pump being in fluidic communication with the air outlet and the air inlet;
an air tube for connecting the air outlet to the air connector of the bedpan; and
a water tube for connecting the water outlet to the liquid connector of the bedpan;
the waste management module comprising:
a vacuum inlet pipe positioned in a sidewall of the module housing, the vacuum inlet pipe curving toward the bottom of the module housing;
a waste inlet pipe positioned in a sidewall of the module housing, the waste inlet pipe curving toward the bottom of the module housing;
a waste tank selectively positionable in the module housing and attachable to the waste inlet pipe and the vacuum inlet pipe;
a waste hose selectively attachable the waste inlet pipe and the waste tube of the bedpan; and
a vacuum hose selectively attachable the vacuum inlet pipe and being configured to attach to the air mover;
a power supply attached to the module housing and operationally engaged to the air pump and the water pump; and
a switch attached to the module housing and operationally engaged to the power supply, the air pump, and the water pump.

17. The waste capturing system of claim 16, further including a filter being positioned in the vacuum inlet pipe such that particulate matter in air passing to the air mover is removed by the filter.

18. The waste capturing system of claim 16, further including a controller operationally being engaged to the water pump and the air pump for selectively actuating the cleaning module.

19. The waste capturing system of claim 18, further including a power outlet attached to the module housing and operationally engaged to the power supply and the controller, such that the air mover can be powered via the power supply and selectively actuated by the controller.

20. A method of managing bodily waste excreted by a bedbound user, the method comprising providing the waste capturing system of claim 12, and:
positioning the bedpan partially under buttocks of a user who is bedbound so that the receptacle is positioned to receive bodily waste from the user;
connecting the waste management module to an air mover;
defecating and/or urinating into the receptacle;
actuating the cleaning module to selectively direct water and air onto the genitals, the perineum, and the anus of the user to rinse and to dry the genitals, the perineum, and the anus; and
urging of the bodily waste and rinse water from the receptacle to the waste management module by passage of air.
